SEQUENCE LISTING (41 sequences)

SEQ ID NO: 1 - protein, 5 aa, Varroa destructor
RYLPT

SEQ ID NO: 2 - protein, 5 aa, Artificial Sequence
  Synthetic Peptide
  MOD_RES (2)..(2): Xaa is Beta-3-Phe, D-Tyr, or Phe(4-F).
  MOD_RES (4)..(4): Xaa is Hyp
RXLXT

SEQ ID NO: 3 - protein, 5 aa, Artificial Sequence
  Synthetic Peptide
  MOD_RES (5)..(5): D-Thr
RYLPT

SEQ ID NO: 4 - protein, 5 aa, Artificial Sequence
  Synthetic Peptide
  MOD_RES (4)..(4): Xaa is Hyp or octahydroindole-2-carboxylic acid
RYLXT

SEQ ID NO: 5 - protein, 5 aa, Artificial Sequence
  Synthetic Peptide
  MOD_RES (1)..(1): ACETYLATION
  MOD_RES (2)..(2): Xaa is Phe(4-NO2)
  MOD_RES (4)..(4): Xaa is Hyp
RXLXT

SEQ ID NO: 6 - protein, 5 aa, Artificial Sequence
  Synthetic Peptide
  MOD_RES (2)..(2): Xaa is Phe(4-F), Phe(4-NO2), or Phe(4-Cl).
RXLPT

SEQ ID NO: 7 - protein, 5 aa, Artificial Sequence
  Synthetic peptide
  MOD_RES (2)..(2): Xaa is Beta-3-Phe
  MOD_RES (4)..(4): Xaa is Hyp
RXLXT

SEQ ID NO: 8 - protein, 5 aa, Artificial Sequence
  Synthetic peptide
  MOD_RES (2)..(2): Xaa is D-Tyr
  MOD_RES (4)..(4): Xaa is Hyp
RXLXT

SEQ ID NO: 9 - protein, 5 aa, Artificial Sequence
  Synthetic peptide
  MOD_RES (2)..(2): Xaa is D-Tyr
  MOD_RES (4)..(4): Xaa is octahydroindole-2-carboxylic acid
RXLXT

SEQ ID NO: 10 - protein, 5 aa, Artificial Sequence
  Synthetic Peptide
AYLPT

SEQ ID NO: 11 - protein, 5 aa, Artificial Sequence
  Synthetic Peptide
RYAPT

SEQ ID NO: 12 - protein, 5 aa, Artificial Sequence
  Synthetic Peptide
RYLAT

SEQ ID NO: 13 - protein, 6 aa, Artificial Sequence
  Synthetic Peptide
GRYLPT

SEQ ID NO: 14 - protein, 6 aa, Artificial Sequence
  Synthetic Peptide
RGYLPT

SEQ ID NO: 15 - protein, 6 aa, Artificial Sequence
  Synthetic Peptide
RYGLPT

SEQ ID NO: 16 - protein, 6 aa, Artificial Sequence
  Synthetic Peptide
RYLGPT

SEQ ID NO: 17 - protein, 6 aa, Artificial Sequence
  Synthetic Peptide
RYLPGT

SEQ ID NO: 18 - protein, 6 aa, Artificial Sequence
  Synthetic Peptide
RYLPTG

SEQ ID NO: 19 - protein, 5 aa, Pediculus humanus
RWLPT

SEQ ID NO: 20 - protein, 5 aa, Artificial Sequence
  Synthetic Peptide
  MOD_RES (4)..(4): D-Pro
RYLPT

SEQ ID NO: 21 - protein, 5 aa, Artificial Sequence
  Synthetic Peptide
  MOD_RES (3)..(3): D-Leu
RYLPT

SEQ ID NO: 22 - protein, 5 aa, Artificial Sequence
  Synthetic Peptide
  MOD_RES (2)..(2): D-Tyr
RYLPT

SEQ ID NO: 23 - protein, 5 aa, Artificial Sequence
  Synthetic Peptide
  MOD_RES (1)..(1): D-Arg
RYLPT

SEQ ID NO: 24 - protein, 5 aa, Artificial Sequence
  Synthetic Peptide
  MOD_RES (4)..(4): Beta-3-Pro
RYLPT

SEQ ID NO: 25 - protein, 5 aa, Artificial Sequence
  Synthetic Peptide
  MOD_RES (3)..(3): Beta-3-Leu
RYLPT

SEQ ID NO: 26 - protein, 5 aa, Artificial Sequence
  Synthetic Peptide
  MOD_RES (2)..(2): Beta-3-Phe
RFLPT

SEQ ID NO: 27 - protein, 5 aa, Artificial Sequence
  Synthetic Peptide
  MOD_RES (4)..(4): Xaa is Hyp
RYLXT

SEQ ID NO: 28 - protein, 5 aa, Artificial Sequence
  Synthetic Peptide
  MOD_RES (4)..(4): Xaa is 2-amino butylic acid
RYLXT

SEQ ID NO: 29 - protein, 5 aa, Artificial Sequence
  Synthetic Peptide
  MOD_RES (4)..(4): Xaa is octahydroindole-2-carboxylic acid
RYLXT

SEQ ID NO: 30 - protein, 5 aa, Artificial Sequence
  Synthetic Peptide
  MOD_RES (1)..(1): ACETYLATION
  MOD_RES (2)..(2): Xaa is Phe(4-F)
RXLPT

SEQ ID NO: 31 - protein, 5 aa, Artificial Sequence
  Synthetic Peptide
  MOD_RES (2)..(2): Xaa is Phe(4-F)
RXLPT

SEQ ID NO: 32 - protein, 5 aa, Artificial Sequence
  Synthetic Peptide
  MOD_RES (2)..(2): Xaa is Phe(4-NO2)
RXLPT

SEQ ID NO: 33 - protein, 5 aa, Artificial Sequence
  Synthetic Peptide
  MOD_RES (2)..(2): Xaa is Phe(4-Cl)
RXLPT

SEQ ID NO: 34 - protein, 5 aa, Artificial Sequence
  Synthetic Peptide
  MOD_RES (2)..(2): Xaa is Phe(4-F)
  MOD_RES (4)..(4): Xaa is Hyp
RXLXT

SEQ ID NO: 35 - protein, 5 aa, Artificial Sequence
  Synthetic Peptide
  MOD_RES (1)..(1): ACETYLATION
  MOD_RES (2)..(2): Xaa is Phe(4-F)
  MOD_RES (4)..(4): Xaa is Hyp
RXLXT

SEQ ID NO: 36 - protein, 5 aa, Oryctes borbonicus
RYLPA

SEQ ID NO: 37 - protein, 5 aa, Artificial Sequence
  Synthetic Peptide
  MOD_RES (1)..(1): ACETYLATION
  MOD_RES (2)..(2): Xaa is Phe(4-Cl)
  MOD_RES (4)..(4): Xaa is Hyp
RXLXT

SEQ ID NO: 38 - protein, 5 aa, Unknown
  Coccinellidae
RYLST

SEQ ID NO: 39 - protein, 5 aa, Leptinotarsa decemlineata
RALPT

SEQ ID NO: 40 - protein, 88 aa, Varroa destructor
MSARGILWGLILVGVILLSLSVESQARYLPTRADPARRERIREILRALLLLSPGEPEARGPYTSSYEYGTGAGDLKGLERSDWDSSST

SEQ ID NO: 41 - protein, 4 aa, Drosophila melanogaster
  MOD_RES (4)..(4): AMIDATION
FMRF

(19) United States
(12) Patent Application Publication
(10) Pub. No.: US 2022/0338471 A1
(43) Pub. Date: Oct. 27, 2022

(54) HONEY BEE-SAFE ACARICIDAL COMPOUNDS

(71) Applicants: Kansas State University Research Foundation, Manhattan, KS (US); The United States of America, as represented by the Secretary of Agriculture, Washington, DC (US); Board of Regents of The University of Nebraska, Lincoln, NE (US)

(72) Inventors: Yoonseong Park, Manhattan, KS (US); Ronald Nachman, College Station, TX (US); Troy Anderson, Lincoln, NE (US)

(21) Appl. No.: 17/762,994

(22) PCT Filed: Sep. 23, 2020
(86) PCT No.: PCT/US2020/052134

§ 371 Date: Mar. 23, 2022

Related U.S. Application Data
(60) Provisional application No. 62/904,436, filed on Sep. 23, 2019.

(51) Int. Cl. A01N 25/32; A01N 25/02; A01N 25/12; A01P 7/00

(52) U.S. Cl. CPC A01N 25/32; A01N 25/02; A01N 25/12; A01P 7/00

(57) ABSTRACT

Described herein are bee-safe acaricides that target the varroa mite-specific neuropeptidergic system regulated by proctolin, and methods of use. The acaricides comprise a peptide analog or peptidomimetic of varroa mite proctolin for selective targeting of this pest in a bee colony. The compounds are also effective against other pests, such as ticks. The peptide analog or peptidomimetic generally comprises one or two amino acid residue substitutions or insertions. Also described herein are methods of screening compounds for activity on a proctolin receptor.

### CROSS-REFERENCE TO RELATED APPLICATIONS

The present application claims the priority benefit of U.S. Provisional Patent Application Ser. No. 62/904,436, filed Sep. 23, 2019, entitled HONEY BEE-SAFE ACARICIDAL COMPOUND DEVELOPED FROM THE BACKBONE OF AMINO ACID SEQUENCE OF PROCTOLIN ARG-TYR-LEU-PRO-THR, which is SEQ ID NO:1, the entire disclosure of which is incorporated by reference in its entirety herein.

### SEQUENCE LISTING

The following application contains a sequence listing in computer readable format (CRF), submitted as a text file in ASCII format entitled “Sequence_Listing,” created on Sep. 22, 2020, as 10 KB. The content of the CRF is hereby incorporated by reference.

### BACKGROUND OF THE INVENTION

### Field of the Invention

The present disclosure is generally directed to compositions and methods for selectively targeting the proctolin neuroendocrine pathway in mites and ticks. These compositions are particularly useful for (although not limited to) targeting such pests, particularly in honey bee colonies without harming the bees. The bioactive peptide analogs of proctolin surprisingly have high activities on the proctolin receptor with acute toxicities on the mites and ticks.

### Description of Related Art

The varroa mite (Varroa destructor) is an obligatory ectoparasite of honey bees. Host expansion of the varroa mite from the Eastern honey bee Apis cerana to the Western honey bee A. mellifera is associated with the worldwide dispersion of the mite in the last century and has caused a major threat to the maintenance of healthy bee colonies. Infestation by varroa mites causes direct damage to the bee colony and facilitates the transmission of pathogens. Control of the varroa mite in beehives has relied heavily upon the use of synthetic and natural acaricides.

The use of chemical acaricides in beehives to control varroa mites requires stringent criteria. Finding selective acaricides that are nontoxic to honey bees is a challenging task because the arachnids and insects belong to the same phylum, Arthropoda, and diverged approximately 750 million years ago. In addition, acaricides and their metabolized forms need to be without adverse effects on human health because of the consumption of honey. Moreover, resistance to acaricide in the mites became another hurdle in control of the Varroa mites.

What is needed are new control methods against ectoparasitic honey bee mites, such as Varroa destructor. Such control methods could also be applicable to other pests, such as ticks.

### SUMMARY OF THE INVENTION

In one embodiment, there is provided a peptide analog or peptidomimetic of proctolin (SEQ ID NO: 1) for controlling parasitic mites and/or ticks, particularly in bee colonies. The peptide analog or peptidomimetic comprises, consists essentially, or even consists of SEQ ID NO:1 with one or two amino acid residue substitutions or insertions. In certain embodiments, a bee-safe acaricide is provided comprising the peptide analog or peptidomimetic.

In another embodiment, there is provided a method of controlling parasitic mites and/or ticks, particularly in a bee colony. The method comprises applying an acaricide to the bee colony. The acaricide comprises a peptide analog or peptidomimetic of proctolin (SEQ ID NO: 1) comprising one or two amino acid residue substitutions or insertions.

In another embodiment, there is provided a method of screening compounds for activity on a proctolin receptor. The method comprises providing a cell culture system expressing varroa mite proctolin receptor, and incubating a candidate compound under culture conditions, and detecting binding of the candidate compound to the receptor. Exemplary candidate compounds include peptide analog or peptidomimetic of proctolin (SEQ ID NO: 1) comprising one or two amino acid residue substitutions or insertions. Analysis can also be performed, for example, to determine the inhibitory activity, e.g., EC₅₀, of the peptide analog or peptidomimetic.

In another embodiment, there is provided the use of a peptide analog or peptidomimetic of proctolin (SEQ ID NO: 1) for controlling parasitic mites in bee colonies. The peptide analog or peptidomimetic comprises one or two amino acid residue substitutions or insertions.

### BRIEF DESCRIPTION OF THE DRAWINGS

FIG. 1 shows the distribution of proctolin and proctolin receptors in arthropods, wherein the tree shows the cluster of proctolin receptors in arthropods is closely related to the FMRFamide (H-Phe-Met-Arg-Phe-NH₂) (SEQ ID NO: 41) receptors and MIP (myoinhibitory peptide) receptors, with solid circles for the bootstrapping value higher than 80 and empty circles for higher than 50%;

FIG. 2 is a set of graphs showing dose-response curve for proctolin, its analogs, and peptidomimetics, wherein the proctolin EC₅₀ is 0.22 nM;

FIG. 3 is a graph showing dose-response curve for proctolin, its analogs, and peptidomimetics;

FIG. 4 is a set of graphs showing time-dependent efficacy of peptide mimics to varroa mites; and

FIG. 5 is a set of graphs showing time-dependent efficacy to varroa mites of the same peptide mimic as FIG. 4 but using different concentrations.

### DETAILED DESCRIPTION

Bee-safe acaricides are described herein, which target the varroa mite-specific neuropeptidergic system regulated by proctolin. Proctolin is a pentapeptide with the highly conserved sequence RYLPT (SEQ ID NO: 1), known to act through a G protein-coupled receptor to elicit myotropic activity. Moths and bees and vertebrates (e.g., humans) lack both proctolin and its receptor. As such, it provides a selective target for controlling, eliminating, decreasing, inhibiting, and/or treating varroa mite infestation in a bee colony, without harming the bees and humans who consume honey.

Also described herein is an assay system using the varroa mite proctolin receptor which can be used for high throughput screening of chemical libraries. Peptidomimetics were shown to retain strong activity on the varroa mite proctolin receptor and provided acute toxicity on the varroa mite, offering guidelines for the development of analogs of higher activity and bioavailability. In particular embodiments, analogs that feature double replacements (substitutions) of the amino acids surprisingly offer enhanced peptidase protection to all peptide bonds and retain full efficacy and significant retention of potency and represent promising lead analogs.

In addition to the use of the developed compound for varroa mite control, embodiments of the invention are directed to a general acaricidal compound, which is otherwise harmless to other organisms. Target pest species include, but are not limited to, varroa mite, honey bee tracheal mite in apiculture, grain mite, mold mite, and cheese mite in stored products, spider mite in agricultural plants, as well as numerous tick species important for animal and human health. Ticks include, for example, Ornithodorus moubata, Ixodes ricinus, Boophilus microplus and Amblyomma hebreum, and mites include, for example, Varroa destructor, Sarcoptes scabiei, Dermanyssus gallinae, Tetranychus urticae, Tetranychus cinnabarinus, and Oligonychus pratensis.

In one or more embodiments, the disclosure concerns a peptide analog or peptidomimetic of proctolin (SEQ ID NO: 1) for controlling parasitic mites in bee colonies. The peptide analog or peptidomimetic generally comprises one or two amino acid residue substitutions or insertions of the proctolin conserved sequence RYLPT (SEQ ID NO: 1). The one or two amino acid residue substitutions or insertions may be made at (substitutions) or between (insertions) any of the R1, Y2, L3, P4, and T5 positions, as well as insertions at the N- and C-terminus of SEQ ID NO: 1. The substitutions or insertions may be made using a variety of different amino acids, including β-amino acids, D-amino acids, and modified residues.

In certain embodiments, the peptide analog or peptidomimetic comprises at least one amino acid residue substitution at the L3, P4, or T5 position of SEQ ID NO: 1. In certain embodiments, the amino acid residue substitution at the L3, P4, or T5 comprises an alanine substitution. In certain embodiments, the peptide analog or peptidomimetic comprises an amino acid residue substitution at the P4 position of SEQ ID NO: 1. In certain embodiments, the amino acid residue substitution at the P4 position comprises a residue selected from the group consisting of β-amino acids, D-amino acid, modified prolines (e.g., hydroxyproline), octahydroindole-2-carboxylic acid, and aminoisobutyric acid. In certain embodiments, the peptide analog or peptidomimetic comprises a substitution at the R1 position of SEQ ID NO: 1. In certain embodiments, the amino acid residue substitution at the R1 position comprises a D-amino acid.

In certain embodiments, the peptide analog or peptidomimetic comprises an amino acid residue substitution at the Y2 position of SEQ ID NO: 1. This substitution may be made independently (i.e., the only modification to SEQ ID NO: 1) or in combination with one or more modifications described above. In certain embodiments, the substitution at the Y2 position comprises a phenylalanine substitution (carrying the aromatic ring). In certain such embodiments, the substitution at the Y2 position comprises a phenylalanine with a ring modification selected from the group consisting of 4-F, 4-NO₂, and 4-Cl.

In certain embodiments, the peptide analog or peptidomimetic comprises two amino acid residue substitutions. In certain embodiments, the two amino acid residue substitutions are at the Y2 and P4 positions of SEQ ID NO: 1. In a particularly preferred embodiments, the peptide analog or peptidomimetic comprises a D-amino acid or β-amino acid substitution at the Y2 position and a modified proline (e.g., hydroxyproline) substitution at the P4 position of SEQ ID NO: 1. In another particularly preferred embodiment, the peptide analog or peptidomimetic comprises a phenylalanine substitution (or modified phenylalanine) at the Y2 position and a hydroxyproline substitution at the P4 position of SEQ ID NO: 1.

In certain embodiments, the peptide analog or peptidomimetic comprises at least one amino acid residue insertion. In certain embodiments, the amino acid residue insertion comprises a glycine insertion. In certain such embodiments, the glycine residue insertion is at the N-terminus of SEQ ID NO: 1. Particularly preferred peptide analog or peptidomimetics are selected from the group consisting of:

(SEQ ID NO: 2)
RX₁LX₂T, where X₁ is B³F, dY, or Phe(4-F), and
X₂ is HyP;
(SEQ ID NO: 3)
RYLP[dT];
(SEQ ID NO: 4)
RYLX₃T, where X₃ is HyP or Oic;
(SEQ ID NO: 5)
Ac-R[Phe(4-NO2)]L[HyP]T;
and
(SEQ ID NO: 6)
RX₄LPT, where X₄ is Phe(4-F), Phe(4-NO2),
or
Phe(4-Cl).

Other preferred peptide analogs or peptidomimetics include those listed in Table 2, below.

In one or more embodiments, the peptide analogs or peptidomimetics described herein are used as a bee-safe acaricide. Bee-safe acaricide compositions may comprise (consist essentially or consist) of a peptide analog or peptidomimetic dispersed or distributed therein in a carrier. The compositions can include a single type of peptide analog or peptidomimetic, or two or more different peptide analogs or peptidomimetics (i.e., mixtures of different peptide analogs or peptidomimetics). It will be appreciated that the particular composition will depend upon whether it is intended to be edible and fed upon by the bees and/or bee larvae, or whether it is intended for application to or ingestion by the target pest (e.g., mites).

The compositions can be in a dry form or in a liquid form. Dry forms includes particulate forms, including powders, granules and flakes/crumbles, as well as larger bodies including compressed tablets, patties, cakes, and the like. Such compositions can be used dry, or mixed with sugar syrup. In certain embodiments, the dry forms can be applied by dusting onto the bee hive. In certain embodiments, the liquid forms can be used for spray applications to the colony or fed to the bees.

In general, the peptide analog or peptidomimetic will be included in the composition at levels to provide a treatment dosage to the bee hive of about 0.001 μM to about 1,000 μM, preferably about 0.01 μM to about 100 μM, and more preferably about 0.1 μM to about 1 μM. The peptide analog or peptidomimetic can be dispersed or distribute carrier. Carriers include liquids, such as plant oils (e.g., corn, sunflower, soybean, cottonseed, canola, and the like), as well as water or aqueous solutions. Dry carriers may include lyophilized powders, which may be mixed with inert ingredients. The compositions can also include proteins and/or carbohydrates (e.g., grain flours such as soy, sorghum, wheat, or corn meal), as well as one or more sweetening or flavoring agents (e.g., sucrose, glucose, corn syrup, cane sugar, beet sugar, or sugar alcohols) to attract bees and/or larvae for feeding. Additional inactive binders or excipients can also be included in the composition including cellulose, starches, natural waxes, and the like. The carrier or carrier material as used herein is defined as not including the body of a mite (e.g., Varroa destructor) or tick.

In certain embodiments, the acaricide compositions may be delivered to the mites and/or ticks through the bees, while the bees remain safe from the composition. Accumulation of the compositions in the bee body fat can also deliver the peptide analog or peptidomimetic. The peptide analog or peptidomimetic may also be incorporated into a commercially-available bee supplement or feed substitute, including MegaBee®, Bee-Pro Patties+, Ultra Bee Patties, Nozevit Plus, SuperDFM-HoneyBee, Purina® Hearty Bee™, and the like.

Other compounds (e.g., bee nutrients known in the art) may be added to the composition provided they do not substantially interfere with the intended activity and efficacy of the composition; whether or not a compound interferes with activity and/or efficacy can be determined, for example, by the procedures utilized below.

The compositions can also be used to deliver nutrients, hormones, or other beneficial agents (e.g., brewer's yeast) to bees and/or larvae in the bee colony. In some embodiments, the compositions can also include additional active agents which target one or more colony pests, pathogens, or diseases (e.g., a broad spectrum composition), including antiparasitics (e.g., fumagillin), antibiotics (e.g., tetracycline), and pesticides (e.g., thymol, coumaphos, fluvalinate, pyrethrin, oxalic acid, lactic acid, formic acid, essential oils). Mixture of any of the foregoing active ingredients can be used. In preferred embodiments, except for essential oils, it is preferred that the acaricide compositions are substantially free of the foregoing pesticides, antibiotics, and/or antiparasitics. As used herein, substantially free means that the ingredient is not intentionally incorporated into the formulation, although trace amounts (e.g., less than 0.1% w/w) may be detectable in the composition.

Regardless, the bee-safe acaricide composition is placed in a location where the bees, larvae, and/or the mites will come into contact with the composition. In one or more embodiments, the composition is placed inside the hive, brood box, or other bee colony enclosure associated with the hive. In one or more embodiments, the composition is positioned outside the hive or brood box, but in the vicinity of the hive where bees will otherwise come into contact with the composition (and transport it back to the hive). The compositions can also be paired with a container or structure for containing and/or slowly releasing the composition, such as a paper strip, foam pad or sponge, diffusor, saucer, and the like.

The composition can be used for treatment and/or prophylaxis of a varroa mite infestation, such as by mitigating, reducing, inhibiting, or preventing infestation of varroa mites (i.e., a damaging number of mites in a hive). This is achieved by mitigating, reducing, inhibiting, or decreasing varroa mite numbers in the hive, and/or preventing expansion of mite numbers. Thus, the composition can be provided to the hive prophylactically before observable signs of varroa mite infestation. The composition can be used on a continual or seasonable basis to inhibit infestation by varroa mites. The composition can also be provided to a hive therapeutically in response to signs of varroa mite infestation. Again, the composition can be applied in various manners, including directly to varroa mites in the hive or other hive surfaces such that the mites come into contact therewith. The composition can also be formulated for the bees and/or larvae, which act as a vector to transmit the peptide analog or peptidomimetic to the mites. That is, the mites come into contact with the peptide analog or peptidomimetic as they feed on the bees. The therapeutic and/or prophylactic composition can be used in conjunction with additional colony treatments for disease and/pests, such as a those containing antibiotics, miticides, formic acid, nutraceuticals (thymol), etc. for honeybee dysentery, mites, parasites, etc.

Additional aspects of the invention relate to new assays for screening compounds for activity on a proctolin receptor. The methods generally comprise providing a cell culture system expressing proctolin receptor. Candidate compounds are incubated with the culture system under appropriate culture conditions, and the binding (or lack thereof) or activation (or lack thereof) by the candidate compound to the receptor can be detected. Exemplary candidate compounds include peptide analog or peptidomimetic of proctolin. Further analysis can be carried out, including to quantify the inhibitory effect of the compound, such as by determining the EC₅₀ of the compound, as exemplified in the working examples. The molecular target, proctolin receptor, is uniquely lacking in the honey bee and offers an ideal target for developing bee-safe acaricides. Therefore, the assays in accordance with embodiments of the present invention using the proctolin receptor advantageously can be a relatively easy way to move on to high throughput screening of chemical libraries in an industrial format.

It will be appreciated that these compounds are not necessarily limited to use in conjunction with bee hives or colonies, but could be used to target pests having the same molecular target, proctolin receptor, such as ticks, in other settings besides beekeeping. Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### Examples

The following examples set forth methods in accordance with the invention. It is to be understood, however, that these examples are provided by way of illustration and nothing therein should be taken as a limitation upon the overall scope of the invention.

### INTRODUCTION

The varroa mite, an obligatory ectoparasite of the honey bee, is a major threat to the maintenance of healthy bee colonies. The use of chemical acaricides in beehives to control varroa mites requires stringent criteria for non-toxicity to honey bees and vertebrates. A biorational approach for the development of bee-safe acaricides is proposed that targets the varroa mite-specific neuropeptidergic system regulated by proctolin. Proctolin is a pentapeptide with the highly conserved sequence RYLPT (SEQ ID NO: 1), known to act through a G protein-coupled receptor to elicit myotropic activity, particularly in many different arthropods. This peptide has thus far been found only in arthropods based on homologous sequences, and are absent in vertebrates. Interestingly, this peptide and the receptor have also not been found in Lepidoptera and Hymenoptera, including the honey bee, providing a highly selective acricidal target.

The varroa mite proctolin receptor was cloned and functionally expressed in this study, and a similar principle to the proctolin neuropeptidergic system was applied. A functionally expressed varroa mite proctolin receptor was tested against a series of proctolin analogs as well as peptidomimetics containing biostability enhancing components. The agonistic activities of 14 different peptidomimetics and 11 peptide variants were evaluated and provide an insight for an understanding of ligand-receptor interactions. A number of peptidomimetics showed strong agonistic activity, which could represent a highly promising approach for the development of bee-safe acaricidal agents for the management of the varroa mites.

### Materials and Methods

Chemicals: Proctolin and the amino acid replacements, alanine scans and glycine insertions analogs were synthesized by Pepmic (Jiangsu, China) and prepared for greater than 85% purity. For Chinese hamster ovarian (CHO) cell culture, DMEM/F12 medium, Fungizone® and penicillin/streptomycin were obtained from Gibco® Cell Culture at Life Technologies (Grand Island, N.Y.). Coelenterazine h was obtained from ATT Bioquest (Sunnyvale, Calif.). Fetal bovine serum (FBS) was obtained from Atlas biologicals (Fort Collins, Colo.).

Bioinformatics and receptor cloning: Proctolin and the receptor searches in GenBank were made by an initial query of the respective protein sequence of Drosophila melanogaster. When the search yielded orthology in various taxa, further expanded searches were made by using queries of closely related taxa. In the case of the proctolin receptor orthology searches, two criteria were used: back blast of the search output against the D. melanogaster database and clustering in the distance tree view. “Lacking” orthology was concluded after searches of the NCBI database for nr proteins, the RefSeq Genome Database, whole-genome shotgun contigs, and expressed sequence tags.

The receptor cDNA was amplified from the total RNA isolated from a pool of 10 adult mites collected from beehives in Manhattan, Kans. Total RNA was isolated by using TRI Reagent (Zymo Research). First-strand cDNA was synthesized by using an ImProm-II™ Reverse Transcription System (Promega), and PCR amplification was performed by Q5® high-fidelity DNA polymerase (New England Biolabs). The PCR product was cloned into pGEM-T-EZ (Promega) and transferred to pcDNA3.1+(Invitrogen) to add the EcoRI cloning site. The sequence was confirmed by Sanger sequencing and submitted to GenBank (accession number MN462557).

A phylogenetic tree was generated using neighbor joining method with 1000 bootstrapping after alignment made by Muscle in the MEGA7.

Receptor activity assay: Cells were transiently transfected with pcDNA3.1+ containing the open reading frame of varroa mite proctolin receptor (VdProctR) or empty vector using TransFr-2020 (Mirus Bio). Approximately thirty hours after transfection, the cells were collected and preincubated with coelenterazine h for the functional assay. The ligands in serial dilutions were loaded into 96-well opaque plates. Cells treated with coelenterazine h were injected into each well on an Orion microplate luminometer (Berthold) luminescent plate reader. The luminescence was measured for 20 seconds immediately after the cell injections. Data analyses were performed to determine the accumulated luminescence value for 20 seconds after extraction of blank-well luminescence. A dose-response curve was generated and the EC₅₀ (50% effective concentration) was calculated in Origin 7 (OriginLab).

Varroa mite toxicity test: Adult female Varroa mites were collected using a sugar roll method from honey bee hives located at the East Campus apiary of the University of Nebraska (Lincoln, Nebr. USA). The hives were not treated with chemicals for honey bee parasites or pathogens. The mites were sorted in the laboratory with a paint brush and tested within 4 h post collection. The mites were placed in 9 cm diameter Petri dishes (Fisher Scientific) lined with filter paper. The dorsal side of the mites was secured to a strip of tape placed on the filter paper. Ten mites were secured to the tape in each dish and three dishes were used for each treatment (i.e., 30 mites/treatment). The peptide mimics were prepared in autoclaved ddH₂O to a concentration of 0.1 nM for 2334, 2336, and Ac-2442 and 3 nM for 2326. Each peptide mimic was delivered as a 200 nL aliquot to the gnathosoma of the mites with autoclaved ddH₂O serving as the untreated control. Following exposure to the peptide mimics, the number of affected (i.e., dead and/paralyzed) was observed and recorded at 1, 2, 6, 12, 24, and 48 h. The percent effect for each treatment was analyzed using a one-way analysis of variance (ANOVA) with a Tukey's post-hoc test (GraphPad Prism, La Jolla, Calif.; p<0.05).

For injection experiments, ˜10 nL of 1 mM concentrations of the compound 2334 were injected while controls were injections of equal volume of H₂O (n=4 in each). Micro-glass electrodes were used for the injections using the pressure injection system (Dagan PMI-200). All injected animals were within 5 hr after the collections as described above.

Peptidomimetic analog synthesis: Analogs were synthesized on an ABI 433A peptide synthesizer with a modified FastMoc 0.25 procedure using an Fmoc-strategy starting from Rink amide resin (Novabiochem, San Diego, Calif., 0.5 mM/g). The Fmoc protecting group was removed by 20% 4-methyl piperidine in DMF (Dimethyl formamide). A fourfold excess of the respective Fmoc-amino acids was activated in situ using HBTU (2-(1h-benzotriazol-1-yl)-1,1,3,3-tetramethyluronium hexafluorophosphate) (1 eq.)/HOBt (1-hydroxybenzotriazole) (1 eq.) in NMP (N-methylpyrrolidone) or HATU (2-(7-Aza-1H-Benzotiazole-1-yl)-1,1,3,3-tetramethyluronium hexafluorophosphate) (1 eq.)/HOAt (1-hydroxy-7-azabenzotriazole) (1 eq.) in NMP for Aib and the amino acid immediately following it in the sequence. The coupling reactions were base catalyzed with DIPEA (N,N-diisopropylethylamine) (4 eq.) The amino acid side chain protecting groups were PMC for Arginine and tBu for both Threonine and Tyrosine. Acetylation was accomplished using known methods.

The analogs were cleaved from the resin with side-chain deprotection by treatment with TFA (Trifluoroacetic acid):H₂O:TIS (Triisopropylsilane) (95.5:2.5:2.5 v/v/v) for 1.5 h. The solvents were evaporated by vacuum centrifugation and the analogs were desalted on a Waters Cis Sep Pak cartridge (Milford, Mass.) in preparation for purification by HPLC. The analogs were purified on a Waters Delta-Pak Cis reverse-phase column (8×100 mm, 15 μm particle size, 100 Å pore size) with a Waters 510 HPLC system with detection at 214 nm at ambient temperature. Solvent A=0.1% aqueous trifluoroacetic acid (TFA); Solvent B=80% aqueous acetonitrile containing 0.1% TFA. Initial conditions were 10% B followed by a linear increase to 90% B over 40 min.; flow rate, 2 ml/min. Delta-Pak Cis retention times: 2393, Ac-RYLR-IyMT-OH: 3.0 min; 2336, RYL[Oic]T-OH: 3.0 min; 2330, [dR]YLPT-OH: 2.4 min; 2331, RYL[β³P]T-OH: 2.8 min; 2335, RYL[Aib]T-OH: 2.4 min; 2337, 11[dY]L[Hyp]T-OH: 2.5 min; 2327, RYL[dP]T-OH: 2.6 min; 2328, RY[dL]PT-OH: 2.6 min; 2326, RYLP[dT]-OH: 2.1 min; 2329, R[dY]LPT-OH: 2.4 min; 2332, RY[β³L]PT-OH: 2.5 min; 2333, R[β³F]LPT-OH: 2.5 min; 2334, RYL[Hyp]T-OH: 2.4 min; 2340, R[dY]L[Oic]T-OH: 2.4 min.

The analogs were further purified on a Waters Protein Pak 1125 column (7.8×300 mm). Conditions: isocratic using 80% acetonitrile containing 0.1% TFA; flow rate, 2 ml/min. Waters Protein Pak retention times: 2393, Ac-RYL[Hyp]T-OH: 7.0 min; 2336, RYL[Oic]T-OH: 7.0 min; 2330, [dR]YLPT-OH: 7.0 min; 2331, RYL[β³P]T-OH: 7.0 min; 2335, RYL[Aib]T-OH: 7.0 min; 2337, R[dY]L[Hyp]T-OH: 7.5 min; 2327, RYL[dP]T-OH: 7.0 min; 2328, RY[dL]PT-OH: 7.5 min; 2326, RYLP[dT]-OH: 7.5 min; 2329, R[dY]LPT-OH: 7.0 min; 2332, RY[β³L]PT-OH: 6.5 min; 2333, R[β³F]LPT-OH: 6.5 min; 2334, RYL[Hyp]T-OH: 7.5 min; 2340, R[dY]L[Oic]T-OH: 7.5 min.

Amino acid analysis was carried out to quantify the analogs and to confirm identity: 2393, Ac-RYL[Hyp]T-OH: L[1.0], R[1.0], T[1.0], Y[1.0]; 2336, RYL[Oic]T-OH: L[1.0], R[1.0], T[1.0], Y[1.1]; 2330, [dR]YLPT-OH: L[1.0], P[1.2], R[1.0], T[1.0], Y[1.1]; 2331, RYL[β³P]T-OH: L[1.0], R[1.0], T[1.0], Y[1.1]; 2335, RYL[Aib]T-OH: L[1.0], R[0.9], T[1.0], Y[1.1]; 2337, R[dY]L[Hyp]T-OH: L[1.0], R[0.9], T[1.0], Y[1.1]; 2327, RYL[dP]T-OH: L[1.0], P[1.0], R[1.0], T[1.0], Y[1.0]; 2328, RY[dL]PT-OH: L[1.0], P[1.0], R[1.0], T[1.0], Y[1.0]; 2326, RYLP[dT]-OH: L[1.0], P[1.0], R[1.0], T[1.0], Y[1.1]; 2329, R[dY]LPT-OH: L[1.0], P[1.0], R[1.0], T[1.0], Y[1.1]; 2332, RY[β³L]PT-OH: P[1.1], R[1.0], T[1.0], Y[1.0]; 2333, R[β³F]LPT-OH: L[1.0], P[0.9], R[0.9], T[1.0]; 2334, RYL[Hyp]T-OH: L[1.0], R[1.0], T[1.1], Y[1.0]; 2340, R[dY]L[Oic]T-OH: L[1.0], R[1.0], T[1.1], Y[1.0]. The identity of the analogs was also confirmed by MALDI-MS on a Kratos Kompact Probe MALDI-MS instrument (Shimadzu, Columbia, Md.). The following molecular ions (MH⁺) were observed: 2393, Ac-RYL[Hyp]T-OH: 708.1 (calc. 708.0); 2336, RYL[Oic]T-OH: 704.8 (calc. 703.9); 2330, [dR]YLPT-OH: 649.8 (calc. 649.7); 2331, RYL[β3P]T-OH: 663.6 (calc. 663.0); 2335, RYL[Aib]T-OH: 637.6 (calc. 637.7); 2337, R[dY]L[Hyp]T-OH: 665.6 (calc. 655.8); 2327, RYL[dP]T-OH: 649.7 (calc. 649.7); 2328, RY[dL]PT-OH: 649.7 (calc. 649.7); 2326, RYLP[dT]-OH: 649.3 (calc. 649.0); 2329, R[dY]LPT-OH: 649.7 (calc. 649.7); 2332, RY[β³L]PT-OH: 663.5 (calc. 663.0); 2333, R[β³F]LPT-OH: 647.4 (calc. 647.0); 2334, RYL[Hyp]T-OH: 665.8 (calc. 665.7); 2340, R[dY]L[Oic]T-OH: 703.7 (calc. 703.8).

### Results and Discussion

### Arachnid Proctolin and the G Protein-Coupled Receptor

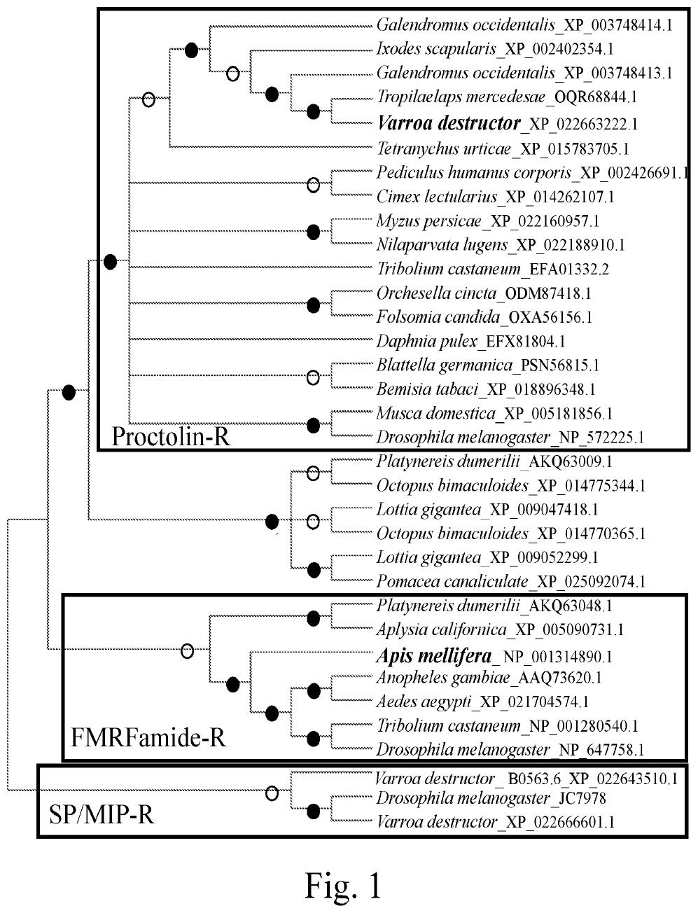

Proctolin and its receptors are widely distributed in arthropods, including insects, crustaceans, and arachnids (FIG. 1). The varroa mite gene encoding proctolin has a short open reading frame with a predicted N-terminal signal peptide. Immediately after the canonical signal peptide, the proctolin sequence followed and ended with a single R, a presumed monobasic cleavage site. See below, the proctolin amino acid sequence in Varroa destructor based on the genome sequence, with underline for signal peptide and bold and highlighted RYLPT (SEQ ID NO:1) is the predicted mature peptide:

(SEQ ID NO: 40)
MSARGILWGLILVGVILLSLSVESQARYLPTRADPARRERIREILRALL
LLSPGEPEARGPYTSSYEYGTGAGDLKGLER SDWDSSST

In a survey of genes encoding proctolin in other taxa, almost all proctolin sequences identified in the search contained a strictly conserved pentapeptide followed by an R for monobasic cleavage with few exceptions.

The human louse (Pediculus humanus) contains the sequence RWLPT (SEQ ID NO: 19), where the second aromatic amino acid W2 replaced the aromatic Y2 in the consensus sequence. The rhinoceros beetle (Oryctes borbonicus) has RYLPA (SEQ ID NO: 36) featuring a replacement of T5 with A5. Lady beetles (Coccinellidae) encode for RYLST (SEQ ID NO: 38), replacing P4 with S4. A previous study reported a RALPT (SEQ ID NO: 39) variant (replacing Y2 with A2) in addition to the presence of the typical RYLPT (SEQ ID NO: 1) in Colorado potato beetle (Leptinotarsa decemlineata). However, a search of the Colorado potato beetle genome identified only RYLPT (SEQ ID NO: 1), and the Y2 to A2 replacement was not identified in the Blast search algorithm (PAM30, E-value threshold: 100). Overall, natural variations of proctolin were found, but with a limited number of cases involving the 4^{th} or 5^{th} amino acid residues, while W2 replacement retains the aromatic side chain in the tryptophan.

A proctolin receptor was first identified in D. melanogaster while the peptide proctolin was first described in the American cockroach in 1975. In a phylogenetic analysis and in Blast searches for the proctolin receptor, an FMRFamide receptor was found to be the closest to the proctolin receptor group. The sex peptide receptor (also known as the myoinhibitory peptide or allatostatin B receptor) was the next closest group of G protein-coupled receptors (See Table 1, below). Interestingly, analysis of the basal lineages of Bilateria, lophotrochozoans including mollusks and annelids, revealed that a group of G-protein coupled receptors were closely related to and grouped with the proctolin receptor of arthropods. Proctolin-like sequences in these taxa were not found in our blast searches. The authentic ligand for this group of receptors remains to be uncovered.

TABLE 1
Results of GenBank search showing the presence or absence
of the sequences in different groups of taxa.

| | Proctolin | Proctolin R |
|---|---|---|
| Hexapoda | | |
| Diptera | yes | yes |
| Culicidae | — | — |
| Lepidoptera | — | — |
| Coleoptera | yes | yes |
| Hymenoptera | — | — |
| Hemiptera | yes | yes |
| Phthiraptera | yes | yes |
| Thysanoptera | yes | yes |
| Blattodea | yes¹ | yes |
| Isoptera | yes | yes |
| Orthoptera | —² | yes³ |
| Collembola | yes | yes |
| Crustacea | yes³ | yes |
| Arachnida | yes | yes |

¹Proctolin in Zootermopsis nevadensis was identified, while other species in this order of insects carry only partial sequences (see more details in the text).
²Proctolin sequence was not found in ortholoteran genome sequences.
³Partial proctolin receptor orthology was identified.

In a survey of genome sequences, the proctolin gene was not identified in the insect orders Lepidoptera and Hymenoptera or the family Culicidae of the order Diptera, which includes mosquito species, important vectors of disease in human and animals. These species also lacked proctolin receptor orthologues. Based on the punctuated groups of taxa lacking proctolin and its receptor and the availability of many genome sequences for the species in these groups of insects, it is likely that they truly lack both proctolin and the receptors in independent evolutionary lineages.

In the case of Blattodea, where the first proctolin was isolated, the proctolin sequence was identified only in the genome sequence of Zootermopsis nevadensis (Blattodea; Isoptera). And, only a part of the prepropeptide encoding C-terminal amino acids lacking the N-terminal part of the mature proctolin sequence was identified in Periplaneta americana, Blattella germanica, and Cryptotermes secundus. Proctolin receptors were also found in Periplaneta americana, and in C. secundus (Blattodea; Isoptera). The searches in the multiple species in Orthoptera genomes found similar results. The proctolin sequence was not identified in the genome sequences of Orthoptera, although proctolin was extensively studied in locust. Only a partial sequence covering the receptor ortholog was identified in Laupala kohalensis and Locusta migratoria. The identification of only partial sequences of proctolin genes and its receptor in Blattodea and Orthoptera is likely due to incomplete genome sequences and annotations, suggesting that these taxa likely retain the proctolin signaling pathway.

A number of previous reports contradicted what was found in the genome sequence survey, i.e., the lack of a proctolin system in the Orders Lepidoptera and Hymenoptera. Proctolin immunoreactivity has been described in the gypsy moth and Manduca sexta. A chromatographic isolation followed by cockroach hindgut bioassay described extremely low quantities of proctolin in honey bee and tent moth (Malacosoma americana): 0.12 and 0.17 mg/kg of tissue, respectively. Proctolin activity on the Bombyx mori larval gut has been previously found, though an extremely high concentration of 180 μM is required for hindgut contraction, and 10 μM is required in the midgut to slightly reduce leucine uptake. In the honey bee, an extremely high dose of proctolin delivered via injection, 1 μL containing 1 μg (1.54 mmol), increased the egg-laying activity of the queen. However, in these studies, the bioactivity was very weak with extremely high concentrations of proctolin required for the bioactivities. The bioactivities described in these studies are unlikely a consequence of the natural endogenous bioactivities of proctolin.

### Analogs and Peptidomimetics

The receptor expressed in the heterologous expression system showed strong reactivity to proctolin, exhibiting an EC₅₀ of 0.22 nM. The negative control, transfection of the empty vector, did not show any activity at a high concentration of 10 μM proctolin. The receptor was also tested with a variety of peptide analogues and peptidomimetics.

An alanine scan series of analogues was evaluated to determine the importance of the side chains of specific amino acid residues in SEQ ID NO:1. The first two amino acids R1 and Y2 are surprisingly the most important, while the fourth residue P4 can be replaced by A with surprisingly only minor loss of the activity of the receptor. The replacement of the third and fifth amino acids (L3 and T5, respectively) to A resulted in moderately reduced activity. Therefore, the first two amino acids R and Y likely contain side chains that interact strongly with the receptor.

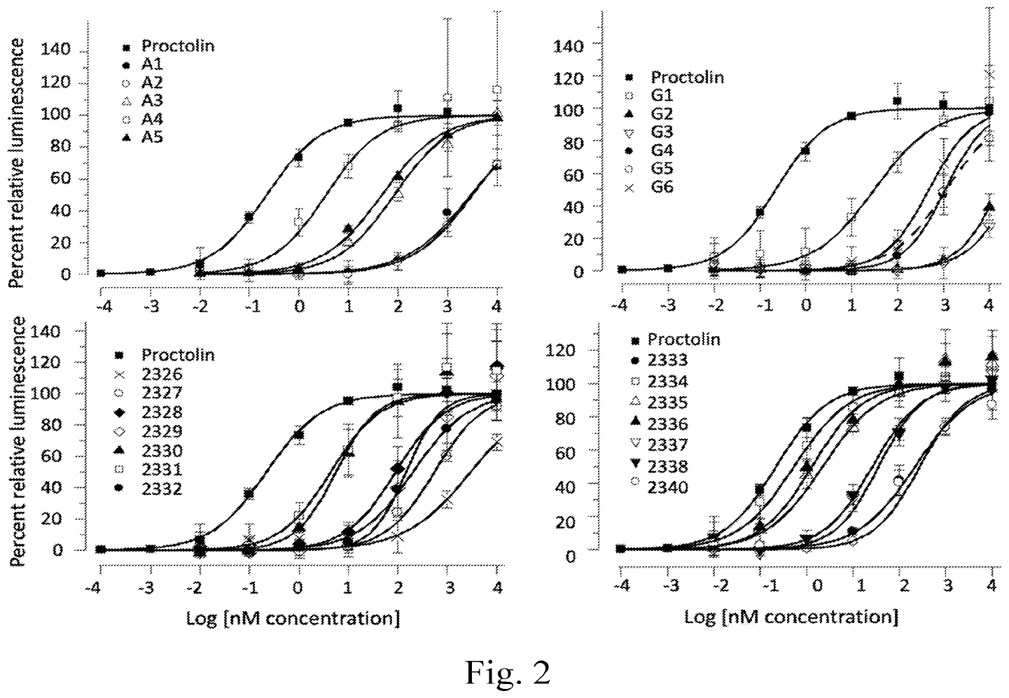

Glycine insertions were used to examine the importance of any two consecutive amino acid residues. The insertion of G at the position between R1 and Y2 and between Y2 and L3 dramatically reduced the activity (Table 2 and FIG. 2). All other G insertions resulted in significant reductions in activity except the N-terminus attachment of G, which surprisingly resulted in a minor loss in activity (Table 2 and FIG. 2). Without being bound by any theory, the space in the N-terminus may allow chemical modification that offers increased permeability and blocks the aminopeptidase N-mediated degradation of the proctolin peptidomimetics.

A series of peptidomimetic analogs of proctolin were chosen for synthesis and receptor evaluation that incorporated components that can enhance resistance to peptidase enzyme hydrolysis, i.e., biostability (including such enzymes as ACE, neprilysin, and aminopeptidases). The first set of five analogs involved sequential replacement of each of the amino acid residues with a D-amino acid. D-amino acids are not recognized by peptidase enzymes and can confer peptidase resistance to adjacent peptide bonds. Most D-amino acid replacements led to significant loss of potency, with the major exception being surprisingly the replacement of the N-terminal R with dR in 2330, which retained activity at 5.6 nM for the EC₅₀. This modification is of importance as it can protect the N-terminus from aminopeptidase degradation.

A second set of analogs involved the replacement of amino acid residues with β-amino acids; which are also not recognized by peptidases and can confer resistance to adjacent peptide bonds to enzyme hydrolysis. The amino acids chosen for replacement depended on the commercial availability of appropriate j-amino acids. Thus, L and P were replaced with β³L (2332) and β³P (2331), respectively. Finally, the aromatic Y residue was replaced with aromatic β³F (2333). Of these three analogs, analog 2331 (β³P for P) surprisingly retained high potency with an EC₅₀ of 4.4 nM.

The P (Pro) amino acid is an important residue for the stabilization of secondary structures such as β-turns, and this property was therefore retained in each of the members of the next series of analogs. Here, the P was replaced with either a modified, sterically-hindered proline analogs hydroxyproline (Hyp) and octahydroindole-2-carboxylic acid (Oic), or the sterically-hindered, turn-promoting residue aminoisobutyric acid (Aib). One analog in this set also incorporates an acetyl group (Ac) at the N-terminus to increase resistance to aminopeptidase attack. As observed with the high activity retained by analog 2331 (β³P for P), surprisingly these P4 replacement analogs similarly demonstrated high potency retention. Analog 2334 (with Hyp) exhibited high potency with an EC₅₀ of 0.54 nM, only 2.5 times less potent than the natural peptide. Analogs 2336 (with Oic) and 2335 (with Aib) also retained relatively strong potencies in the nanomolar range, with EC₅₀ values of 1.3 and 2.2 nM, respectively. These sterically-hindered Pro replacements can surprisingly confer greater biostability to the analogs that incorporate them.

Additional analogs featured double-replacement analogs to potentially confer enhanced peptidase resistance to every peptide bond in the proctolin sequence. A component that enhances stability is adjacent to each peptide bond in the sequence of this analog pair. In this pair, sterically-hindered P replacements were coupled with either a replacement of Y with dY (2337 or 2340) or with β³F (2338). Despite the major modifications incorporated into analogs 2338 (RX₁LX₂T) (SEQ ID NO: 7, where X₁ is β³F and X₂ is Hyp) and 2337 (RX₁LX₂T) (SEQ ID NO: 8, where X₁ is dY and X₂ is Hyp), the two retained full efficacy (maximal response) and a relatively strong potency with EC₅₀ values of 26 and 36 nM, respectively. Indeed, surprisingly in each case the potency of these double-replacement analogs at positions Y2 and P4 exceeded that of the corresponding analog (2333 and 2329, respectively) with only a single replacement at the Y2 position by a factor of 8 and 4, respectively. Analog 2340 (RX₁LX₂T) (SEQ ID NO: 9, where X₁ is dY and X₂ is Oic) was less potent than 2337 and 2338 by approximately an order of magnitude. Mimetic analogs featuring enhanced biostability have an advantage over native peptides in that they can exist and remain active for a longer period in the hemolymph. The analogs 2338 and 2337 may provide leads for biostable mimetic analogs that can disrupt the processes mediated by proctolin in parasitic mites.

The last set of peptidomimetic tested were the proctolin variants at the Y2 position with phenylalanine carrying the aromatic ring with 4-F, 4-NO2, 4-Cl, and with additional variations for [HyP] replacing at the 4th P and for Ac at the N-termini. This set of peptidomimetics surprisingly showed high activities including hyper activities that have higher activities than endogenous proctolin. Specifically, replacement of the Y2 to [Phe(4-NO₂)], [Phe(4-Cl)], [Phe(4-F)], or [Phe(4-F)] surprisingly showed even higher activities than proctolin. A double-replacement, R[Phe(4-F)]L[HyP]T, also surprisingly had higher activity than proctolin, while it had slightly reduced activity than the single replacement R[Phe(4-F)]LPT (Table 2).

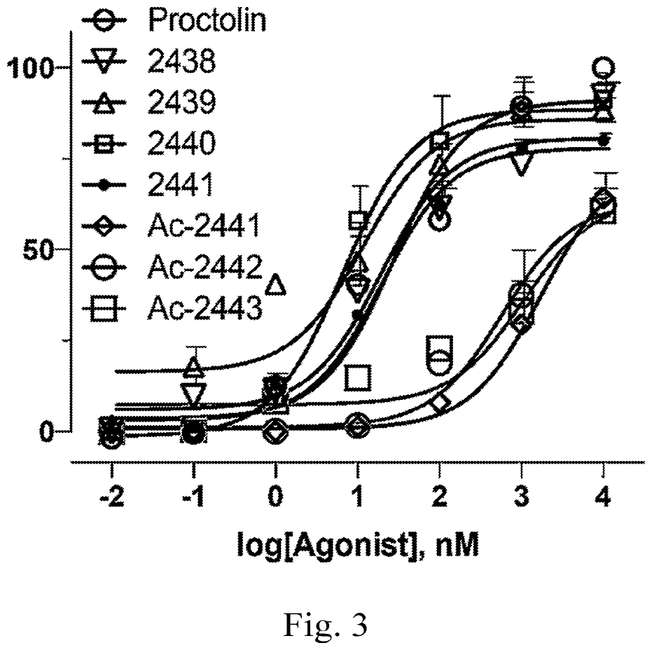

An interesting observation in this study is that the highly active ligands surprisingly often display higher efficacy (maximal response) than proctolin at high concentrations (FIG. 2). A4, G6, 2330, 2331, 2334, and 2336 are in this category; the activities of these ligands at the 1 μM and 10 μM were higher than that of proctolin. The highly active ligands in this study may also have influenced receptor activation of the proctolin receptor in a similar fashion. Certain configurations of the ligand may elicit more efficient conformational changes in the receptor than the endogenous ligand, despite featuring lower binding kinetics. Additional testing of peptidomimetics is shown in FIG. 3 (and Table 2).

### Peptidomimetics Toxicity to Varroa Mites and to Honey Bee

Four peptidomimetics tested for the bioassays were three strong agonists 2334, 2336, and Ac-2442, and an inactive agonist 2326 determined on the Varroa mite proctolin receptor. Initially, the highly active agonist 2334 on the receptor was directly injected into the body cavity (˜5 nL of 200 μM). The injection immediately induced excretion of a drop of fecal material through the anus (3/4 individuals) while the H₂O control did not cause excretion or other noticeable change (0/4). This result indicates that the Varroa mite proctolin activity includes activation of excretory system which is a well-documented activity in other insects.

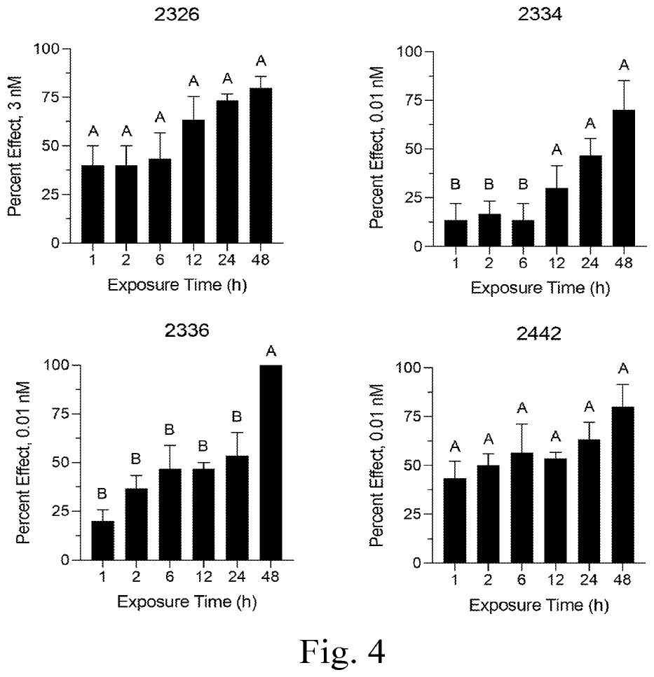

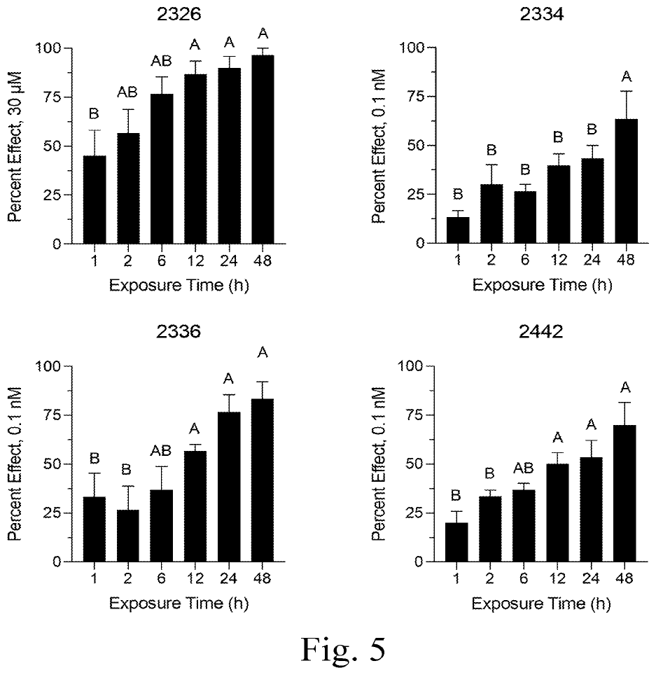

In the continued bioassay (FIG. 4 and FIG. 5), the toxicities were assessed by counting paralysis/death in feeding assay. A drop of 200 nL solution was placed on the gnathosoma of immobilized mite for forced feedings of active components. Low levels of acute toxicities of peptidomimetics were indicated by lower than 50% effects within 6 hours after the treatments. The toxic effects were increased over time and reached 100% of the effects in the 48-hour treatments of 2336. In H₂O control, about 10% or less number of individuals were paralyzed/dead.

In the results shown in FIG. 4 and FIG. 5, female varroa mites were topically treated 2326 at 3 nM (FIG. 4) or 30 μM (FIG. 5) and 2334, 2336, and 2442 at 0.01 nM (FIG. 4) of 0.1 nM (FIG. 5), and the number of mites affected (i.e., paralyzed or dead) was recorded for 1 to 48 h post-treatment. Vertical bars represent the mean±standard error. Different letters above the bars indicate a significant difference between the mean percent effect at each time point using a one-way ANOVA with a Tukey's post-hoc test (p<0.05).

Activities of the proctolin peptidomimetics were tested on the honey bee FMRFamide receptor that is ancestrally related to the proctolin receptor (FIG. 1). Honey bee FMRFamide receptor was cloned and expressed in the same system as described for the procotolin receptor. Proctolin and its peptidomimetics showed no activities while the honey bee FMRFamides showed strong activities on the honey bee FMRFamide receptor. Potential effects of the peptidomimetics 2439, 2334, 2336, Ac-2442, and H₂O control were tested by injections of the compounds into the thorax of worker honey bees. Injections of 50 nL solutions at the 200 μM concentrations did not show any negative effects in 24-hour observations after the injections (n=4 for each compound).

### CONCLUSIONS

In conclusion, this study has successfully developed an assay system using the varroa mite proctolin receptor. Peptidomimetics were shown to retain relatively strong activity on the varroa mite receptor and provide guidelines for the development of 2^{nd} generation analogs of higher activity and bioavailability. In particular, two analogs that feature double replacements offer enhanced peptidase protection to all peptide bonds and retain full efficacy and significant retention of potency and represent promising lead analogs. The peptidomimetics pre-selected by the receptor assays showed significant activities in Varroa mite feeding assays at very low concentrations while high concentrations of the peptidomimetics in honey bee found no detectable adverse effects. And finally, a new approach to developing selective acaricidal compounds targeting a varroa mite specific neuropeptidergic system is outlined in this study.

TABLE 2
Activities of proctolin and its mimetics on the varroa mite proctolin receptor.
D is for D-amino acid, β-3- is for beta -3 amino acids, HyP is for hydroxy
proline, Aib is for 2-amino butylic acid, and Oic is for octahydroindole-2-
carboxylic acid.
A. First set of experiments

| Chemical ID | Sequence | SEQ ID NO: | EC50 (nM) | Fold change |
|---|---|---|---|---|
| Proctolin | RYLPT | 1 | 0.22 | 1.00 |
| | Alanine scan | | | |
| A1 | AYLPT | 10 | 2972.00 | 13509.09 |
| A2 | RALPT | 39 | 3187.00 | 14486.36 |
| A3 | RYAPT | 11 | 84.27 | 383.05 |
| A4 | RYLAT | 12 | 3.54 | 16.09 |
| A5 | RYLPA | 36 | 47.44 | 215.64 |
| | Glycine insert | | | |
| G1 | GRYLPT | 13 | 30.88 | 140.36 |
| G2 | RGYLPT | 14 | 15861.00 | 72095.45 |
| G3 | RYGLPT | 15 | 31431.00 | 142868.18 |
| G4 | RYLGPT | 16 | 1090.00 | 4954.55 |
| G5 | RYLPGT | 17 | 1157.00 | 5259.09 |
| G6 | RYLPTG | 18 | 478.50 | 2175.00 |
| | Peptidomimetics | | | |
| 2326 | RYLP[dT] | 3 | 2992.00 | 13600.00 |
| 2327 | RYL[dP]T | 20 | 549.30 | 2496.82 |
| 2328 | RY[dL]PT | 21 | 90.94 | 413.36 |
| 2329 | R[dY]LPT | 22 | 147.90 | 672.27 |
| 2330 | [dR]YLPT | 23 | 5.62 | 25.55 |
| 2331 | RYL[β3P]T | 24 | 4.40 | 20.00 |
| 2332 | RY[β3L]PT | 25 | 204.00 | 927.27 |
| 2333 | R[β3F]LPT | 26 | 218.20 | 991.82 |
| 2334 | RYL[HyP]T | 27 | 0.54 | 2.45 |
| 2335 | RYL[Aib]T | 28 | 2.22 | 10.09 |
| 2336 | RYL[Oic]T | 29 | 1.31 | 5.95 |
| 2337 | R[dY]L[HyP]T | 8 | 35.86 | 163.00 |
| 2338 | R[β3F]L[HyP]T | 7 | 26.31 | 119.59 |
| 2340 | R[dY]L[Oic]T | 9 | 277.70 | 1262.27 |

A. Second set of peptidomimetics

| | | | | |
|---|---|---|---|---|
| Chemical ID | Sequence | SEQ ID NO: | EC50 (nM) | Fold change |

| | | | | |
|---|---|---|---|---|
| Proctolin | RYLPT | 1 | 14.19* | 1.00 |

| | Peptidomimetics | | |
|---|---|---|---|
| 2393 | Ac-R[Phe(4-F)]LPT | 30 | 73.89 |

| | | | | |
|---|---|---|---|---|
| 2438 | R[Phe(4-F)]LPT | 31 | 7.55 | 0.53 |
| 2439 | R[Phe(4-NO2)]LPT | 32 | 3.15 | 0.22 |
| 2440 | R[Phe(4-Cl)]LPT | 33 | 7.03 | 0.50 |
| 2441 | R[Phe(4-F)]L[HyP]T | 34 | 9.93 | 0.70 |
| Ac-2441 | Ac-R[Phe(4-F)]L[HyP]T | 35 | 471.13 | 33.20 |
| Ac-2442 | Ac-R[Phe(4-NO2)]L[HyP]T | 5 | 98.48 | 6.94 |
| Ac-2443 | Ac-R[Phe(4-Cl)]L[HyP]T | 37 | 108.43 | 7.64 |

*The second set of experiments provided lower EC₅₀ activity for proctolin, which is not unusual in biological replications.

1. A peptide analog or peptidomimetic of proctolin (SEQ ID NO: 1) for controlling parasitic mites and/or ticks, particularly in bee colonies, comprising one or two amino acid residue substitutions or insertions.

2. The peptide analog or peptidomimetic of claim 1, comprising at least one amino acid residue substitution at the L3, P4, or T5 position of SEQ ID NO: 1.

3. The peptide analog or peptidomimetic of claim 1, comprising at least one amino acid residue insertion at the N-terminus of SEQ ID NO: 1.

4. The peptide analog or peptidomimetic of claim 3, wherein the at least one amino acid residue insertion comprises a glycine insertion.

5. The peptide analog or peptidomimetic of claim 1, comprising a D-amino acid substitution at the R1 position of SEQ ID NO: 1.

6. The peptide analog or peptidomimetic of claim 1, comprising a substitution at the P4 position of SEQ ID NO: 1 selected from the group consisting of β-amino acids, hydroxyproline, octahydroindole-2-carboxylic acid, and aminoisobutyric acid.

7. The peptide analog or peptidomimetic of claim 1, comprising an amino acid substitution at the Y2 position of SEQ ID NO: 1.

8. The peptide analog or peptidomimetic of claim 7, wherein the amino acid substitution comprises a phenylalanine substitution.

9. The peptide analog or peptidomimetic of claim 8, wherein the phenylalanine comprises a ring modification selected from the group consisting of 4-F, 4-NO2, and 4-Cl.

10. The peptide analog or peptidomimetic of claim 1, comprising two amino acid residue substitutions.

11. The peptide analog or peptidomimetic of claim 10, wherein the two amino acid residue substitutions are at the Y2 and P4 positions of SEQ ID NO: 1.

12. The peptide analog or peptidomimetic of claim 11, comprising a D-amino acid or β-amino acid substitution at the Y2 position and a hydroxyproline substitution at the P4 position of SEQ ID NO: 1.

13. The peptide analog or peptidomimetic of claim 11, comprising a phenylalanine substitution at the Y2 position and a hydroxyproline substitution at the P4 position of SEQ ID NO: 1.

14. The peptide analog or peptidomimetic of claim 1, selected from the group consisting of:
(SEQ ID NO: 2)
RX₁LX₂T, where X₁ is B³F, dY, or Phe(4-F), and
X₂ is HyP;
(SEQ ID NO: 3)
RYLP[dT];
(SEQ ID NO: 4)
RYLX₃T, where X₃ is HyP or Oic;
(SEQ ID NO: 5)
Ac-R[Phe(4-NO2)]L[HyP]T;
and
(SEQ ID NO: 6)
RX₄LPT, where X₄ is Phe(4-F), Phe(4-NO2),
or
Phe(4-Cl).

15. A bee-safe acaricide composition comprising a peptide analog or peptidomimetic according to claim 1.

16. The bee-safe acaricide composition of claim 15, wherein said peptide analog or peptidomimetic is dispersed in a dry or liquid carrier.

17. A method of controlling parasitic mites and/or ticks, particularly in a bee colony, comprising delivering an acaricide to the bee colony, the acaricide comprising a peptide analog or peptidomimetic according to claim 1.

18. The method of claim 17, wherein said delivering comprises placing said acaricide inside a bee colony enclosure, such as a hive or brood box, or outside the bee colony enclosures proximate to the enclosure where bees will otherwise come into contact with said acaracide.

19. A method of screening compounds for activity on a proctolin receptor comprising:
providing a cell culture system expressing proctolin receptor;
incubating a candidate compound with said cell culture system; and
detecting binding of said candidate compound with said proctolin receptor,
wherein said candidate compound is a peptide analog or peptidomimetic of proctolin (SEQ ID NO: 1) comprising one or two amino acid residue substitutions or insertions.

20. (canceled)
